(19) United States
(12) Patent Application Publication
(10) Pub. No.: US 2026/0287511 A1
(43) Pub. Date: Sep. 24, 2026

(54) GAS ANALYZER APPARATUS

(71) Applicant: ATONARP INC., Minato-ku, Tokyo (JP)

(72) Inventors: Dmitry GORIN, Pleasanton, CA (US); Said BOUMSELLEK, Pleasanton, CA (US); Prakash Sreedhar MURTHY, Tokyo (JP)

(73) Assignee: ATONARP INC., Minato-ku, Tokyo (JP)

(21) Appl. No.: 19/473,260

(22) PCT Filed: Apr. 23, 2024
(86) PCT No.: PCT/JP2024/015949

§ 371 Date: Oct. 7, 2025

Related U.S. Application Data
(60) Provisional application No. 63/498,130, filed on Apr. 25, 2023.

(51) Int. Cl. G01N 21/71; G01N 33/00

(52) U.S. Cl. CPC G01N 21/71; G01N 33/0027

(57) ABSTRACT

A gas analyzer apparatus comprises a sample chamber into which a sample gas to be measured flows, a plasma generator circuit for generate a plasma in the sample chamber, and an analyzer for analyzing the sample gas via the plasma generated in the sample chamber. the plasma generator circuit includes a plasma inducing circuit and an RF power supply circuit that is configured to supply RF power to the plasma inducing circuit. The plasma inducing circuit is integrated directly as at least a part of resonant components of the RF power supply circuit.

### TECHNICAL FIELD

The present invention generally relates to a gas analyzer with a plasma generator.

### BACKGROUND ART

In US2020/0059198, a class-E power oscillator (PO) is disclosed. The class-E PO includes a first inductor, a switch, a first capacitor, a resonant circuit, and a feedback network. The first inductor is coupled in series to a first power supply. The switch is connected between the first inductor and a primary common node. The first capacitor is connected between the first inductor and the primary common node. The resonant circuit includes a second inductor, a second capacitor, and a resistor. The second inductor is connected between the first inductor and the primary common node. The second capacitor is connected between the first inductor and the primary common node, and is coupled in series to the second inductor. The resistor is connected between the first inductor and the primary common node, and is coupled in series to the second inductor. The feedback network is connected between the switch and a feedback node. The feedback node is located between the second inductor and the second capacitor. The feedback network is configured to periodically turn the switch on and off based on a resonance frequency of the resonant circuit.

In WO2020/196452, a gas analyzer apparatus is disclosed. The gas analyzer includes: a sample chamber that is equipped with a dielectric wall structure and into which only sample gas to be measured is introduced; a plasma generation mechanism that generates plasma inside the sample chamber, which has been depressurized, using an electric field and/or a magnetic field applied through the dielectric wall structure; and an analyzer unit that analyzes the sample gas via the generated plasma. By doing so, it is possible to provide a gas analyzer apparatus capable of accurately analyzing sample gases, even those including corrosive gas, over a long period of time.

As shown in FIG. 1, the existing system 101 of generating Rf energy for plasma generation includes a frequency reference source 102, a power amplifier 103, and a plasma-generation load circuit 104 which is connected to the power amplifier by a means of impedance-matched transmission line 105. Parameters of the Plasma-generation load circuit 104 change with the environmental conditions and plasma chamber service creates an impedance mismatch with the transmission line 105 and power amplifier 103. This impedance mismatch limits the performance and service lifetime of the system 101, requiring complicated, expensive additional matching mechanisms of limited reliability and performance range.

### SUMMARY OF INVENTION

One of the challenges is to suppress or prevent impedance mismatch in a plasma generator by using a simple mechanism.

One of the aspects of this invention is a gas analyzer apparatus that comprises: a sample chamber into which a sample gas to be measured flows; a plasma generator circuit configured to generate a plasma in the sample chamber; and an analyzer that is configured to analyze the sample gas via the plasma generated in the sample chamber. The plasma generator circuit includes a plasma inducing circuit that includes at least one of a plasma-inducing coil and a capacitor, and an RF power supply circuit that is configured to supply RF power to the plasma inducing circuit and the plasma inducing circuit is integrated directly as at least a part of resonant components of the RF power supply circuit.

Inventors of this application are attempting to provide a gas analyzer apparatus that can be provided in a very compact form. In such gas analyzer apparatus, the inventors found that by integrating the plasma inducing circuit directly into the RF power supply circuit without a transmission line, not only can the plasma generator circuit be made compact, but the impedance matching mechanism can be eliminated. In addition, because the plasma inducing circuit is coupled (inductively and capacitively) to the plasma chamber (sample chamber), when the plasma chamber parameters change continuously during the normal operation, that causes the effective parameters change of the plasma inducing circuit and feedback directly to the frequency adjusting of the oscillation of the RF power supply circuit without impedance matching mechanism. That makes it possible to supply a self-tuning and self-starting plasma generator circuit. By integrating the plasma inducing circuit as a part of the actual L-C parameters for designing the frequency of the oscillation, the frequency of the oscillation is self-determined by the effective L-C parameters. This self-adjustability of this design allows for not using a special impedance-matching circuitry, and for robustness against any variations of chamber mechanical properties.

The RF power supply circuit may have a class-E power oscillator type. The RF power supply circuit may include: an electrically controlled switch; a first capacitor connected in parallel with the electrically controlled switch; a resonant circuit connected in parallel with the electrically controlled switch that includes the plasma inducing circuit as at least a part of an inductor and a second capacitor, and a third capacitor; and a feedback circuit that is configured to obtain a feedback signal of the electrically controlled switch from a feedback node between the plasma inducing circuit and the third capacitor.

The gas analyzer apparatus may further comprise a base on which the sample chamber, the plasma generator, and the analyzer are assembled as one module to provide as an integrated analyzer module and/or portable or handy analyzer instrument. The gas analyzer apparatus may further comprise an exhaust system that is configured to exhaust from the sample chamber and assembled on the base on which the sample chamber, the plasma generator, and the analyzer are assembled as one module. The sample chamber, the plasma generator, the analyzer, and the exhaust system may be integrated on or into the base as one module. The gas analyzer apparatus may further comprise a first detector that is configured to detect filtered ionized gas from the plasma outputted from the sample chamber. The gas analyzer apparatus may further comprise a second detector configured to analyze the emission of ions in the plasma inside the sample chamber.

Another aspect of this invention is a system that comprises the gas analyzer apparatus, and a sampling device that is configured to supply the sample gas to be measured to the sample chamber. The system may further comprise a process chamber in which a plasma process, such as etching a substrate, CVD (Chemical Vapor Deposition), PVD (Physical Vapor Deposition), or one or more processes related to semiconductor manufacturing, is performed (implemented, carried out) and from which the sample gas is supplied to the gas analyzer apparatus. The compact gas analyzer apparatus of this invention can be installed or mounted on the process equipment, process controller, and/or process monitor easily and economically. The system may further comprise a process monitoring apparatus that monitors at least one process performed (implemented) in the process chamber based on a measurement result of the gas analyzer apparatus. The system may further comprise a process control apparatus that controls at least one process implemented in the process chamber based on a measurement result of the gas analyzer apparatus.

Yet another aspect of this invention is a system that includes a plasma generator chamber and a plasma generator circuit. The plasma generator circuit may include: an electrically controlled switch; a first capacitor connected in parallel with the electrically controlled switch; a resonant circuit connected in parallel with the electrically controlled switch that includes a plasma inducing circuit for generating a plasma in the plasma generator chamber and a third capacitor. The plasma inducing circuit is integrated as at least a part of an inductor and a second capacitor for directly configuring the resonant circuit; and a feedback circuit for obtaining a feedback signal of the electrically controlled switch from a feedback node between the plasma inducing circuit and the third capacitor. The system may further comprise a sampler (sampling line, sampling equipment) that is configured to supply a sample gas to be measured to the plasma generator chamber; and an analyzer that analyzes the sample gas via a plasma generated in the plasma generator chamber. The sampler may be configured to supply the sample gas delivered from a process to be monitored, and the system may further comprise a process monitor for monitoring and controlling the process according to the measurement results of the analyzer.

### BRIEF DESCRIPTION OF DRAWINGS

The embodiments herein will be better understood from the following detailed description with reference to the drawings, in which:

FIG. 1 depicts an example of a prior embodiment of a plasma generator circuit;

FIG. 2 depicts an embodiment of a system including the plasma generator circuit of this invention;

FIG. 3 depicts a diagram of an embodiment of a plasma generator circuit; and

FIG. 4 depicts a reference circuit of a class-E power oscillator (PO).

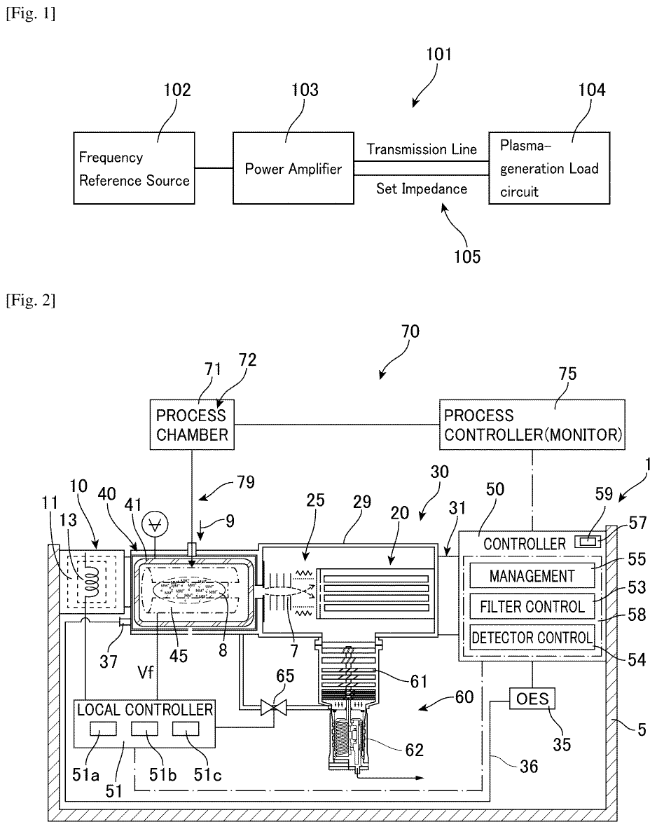

### DESCRIPTION OF EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

FIG. 2 depicts the configuration of a process monitoring system 70 as one example of a system including a gas analyzer apparatus 1. The gas analyzer apparatus 1 analyzes a sample gas 9 supplied from a process chamber 71 in which one or more plasma processes 72 are performed (implemented). The plasma processes 72 performed in process chamber 71 may include a process for forming various types of films or layers on a substrate or a process for etching a substrate, and CVD (Chemical Vapor Deposition) and/or PVD (Physical Vapor Deposition). The plasma process 72 is not limited to a process related to semiconductor manufacturing; it may be a process of laminating various types of thin film on an optical component, such as a lens, a filter, or the like, as a substrate.

As one example, in the field of semiconductors, semiconductor chip structures have become increasingly three-dimensional in recent years due to demands for increased memory capacity, improvements in logic speed, and reduced power consumption. This means that for semiconductor process control, there have been problems with processes becoming more complex, an increased demand for atom-level quality, and an increase in the cost of measurement and monitoring. Monitoring of gas, including reactants and by-products, is essential for process matching, for measurement of transition points during deposition, and for detection of endpoints during etching.

A process monitor (process monitoring apparatus, process monitoring system) 70 includes a process controller (process controller apparatus) 75 that controls the process 72 according to the result of analyzing the sample gas 9, which is supplied from the process chamber 71 where the plasma process 72 is performed, using the gas analyzer apparatus 1. The process controller 75 may include computer resources such as a CPU and memory and may operate according to a control program (program product). The process controller 75 may perform control of one or a plurality of processes 72 performed in the process chamber 71.

In the process monitoring system 70, which uses the gas analyzer apparatus 1 according to the present embodiment, it is possible to provide innovative process control by performing real-time monitoring, even in a harsh environment, and providing highly reliable measurement results. The gas analyzer apparatus 1 functions as a total solution platform developed to dramatically improve the throughput in semiconductor chip manufacturing and maximize the yield rate. As described above, the gas analyzer apparatus 1, according to the present embodiment, is provided as one module with a tiny (an extremely small) installation footprint and can, therefore, be directly connected to the chamber 71 and/or used on site.

The gas analyzer apparatus 1 includes a chamber (sample chamber, plasma generator chamber, sampling chamber) 40 into which a sample gas 9 to be measured flows, a plasma generator circuit 10 that is configured to generate a plasma 8 in the sample chamber 40, and analyzer (analyzer unit) 30 that is configured to analyze the sample gas 9 via the plasma 8 generated in the sample chamber 40. The sample chamber 40 is equipped with a dielectric wall structure 41, and receives an inflow of only the sample gas 9, which is to be measured and is supplied via the sampling device (sampler) 79 from the process chamber 71. The plasma generation circuit 10 includes an RF power supply circuit 11 and a plasma inducing circuit 13 directly integrated as a part of the RF power supply circuit 11. The plasma inducing circuit (plasma-inducing circuit, high frequency supplying mechanism, RF supplying mechanism, RF supplying apparatus) 13 may include an inductance (such as a plasma inducing-coil), an inducing capacitor, or a combination of the inductance and the capacitor for applying a high-frequency electric field and/or magnetic field through (via) the dielectric wall structure 41 to generate the plasma 8 inside the sample chamber 40 that has been depressurized. The apparatus 1 may further comprise a control electrode 45 inside the sample chamber 40 for controlling a floating potential Vf of the plasma 8.

The gas analyzer apparatus 1 according to the present embodiment may be a mass spectrometer type, where the analyzer 30 includes: a filter unit (filter, in the present embodiment, a quadrupole filter) 20 that filters, according to mass-to-charge ratio, ionized sample gas (sample gas ions) 7 generated as the plasma 8 at the sample chamber 40; a focus electrode (ion drawing optical system) 25 that draws in some of the plasma 8 as an ion flow 7; a detector unit (detector, first detector) 31 that detects the filtered ions (ionized gas) from the plasma flow 7; and a vacuum vessel (housing) 29 that houses the filter 20, electrode 25 and the detector 31. The filter 20 may be other types such as an ion trap, or a TOF (time of flight).

The analyzer 30 may include an OES (Optical Emission Spectrometer, second detector) 35 that is configured to analyze the emission of ions in the plasma 8 inside the sample chamber 40. The gas analyzer apparatus 1 includes a light-receiving or light-collecting element 37, such as an objective lens attached to the translucent dielectric wall structure 41 of the sample chamber 40, an optical fiber 36 that guides light from the light-collecting element 37, and a spectroscopic analyzer unit (OES, OES detector apparatus) 35 that spectroscopically analyzes the light supplied by the optical fiber 36. The spectroscopic analyzer of OES 35 may be any detector used in the OES, such as a sequential or multi-channel detector.

The gas analyzer apparatus 1 further includes an exhaust system 60 that keeps the interior of the housing 29 under appropriate negative pressure conditions (vacuum conditions). In the present embodiment, the exhaust system 60 includes a turbo molecular pump (TMP) 61 and a roots pump 62. The exhaust system 60 is a dual-type configuration that also controls the internal pressure of the sample chamber 40 using an intermediate negative pressure stage formed between the TMP 61 and the roots pump 62.

The sample chamber 40, which has been depressurized by the exhaust system 60, receives an inflow, via the sampling device (sampler) 79, of only the sample gas 9 from the process chamber 71, with the plasma 8 being formed by only the sample gas 9 inside the sample chamber 40. The chamber 40 is designed to generate microplasma 8 in an intermediate region, which is neither macroplasma nor nanoplasma. Generating and using microplasma 8 for analyzing is one of the factors for realizing this compact gas analyzer apparatus 1. Examples of the microplasma 8 are plasmas in a region covering sizes of around several millimeters to about 100 ƒÊm. To generate the plasma 8 of this size, the plasma generation circuit 10 generates the plasma 8 for analysis purposes using only the sample gas 9 without using an assist gas (support gas), such as argon gas. The microplasma 8 is small but can supply a sufficient amount of ionized gas 7 to the filter 20 to be detected by the first detector 31, such as Faraday Cup (FC), and has a sufficient amount of volume to be detected by the second detector (OES) 35. The wall body 41 of the sample chamber 40 is composed of a dielectric member (dielectric), and as an example is a dielectric that is highly resistant to plasma, such as quartz, aluminum oxide (Al₂O₃), and silicon nitride (SiN₃).

The sample chamber 40 is a small chamber suited to generating the microplasma 8, for example, the sample chamber 40 may have a total length of 1 to 100 mm and a diameter of 1 to 100 mm. The total length and diameter may be 5 mm or larger, 10 mm or larger, 80 mm or smaller, 50 mm or smaller, or 30 mm or smaller. The capacity of the sample chamber 40 may be 1 mm³ or larger, and/or 105 mm³ or smaller. The capacity of the sample chamber 40 may be 10 mm³ or larger, 30 mm³ or larger, or 100 mm³ or larger. The capacity of the sample chamber 40 may be 104 mm³ or smaller, or 103 mm³ or smaller. In a space of this size, it is easy to control the potential (electric field) inside the space of chamber using the electrode 45 disposed in the chamber. This small sample chamber 40 is one of the merits for providing this compact and modularly structured gas analyzer apparatus 1. In this gas analyzer apparatus 1, corresponding to this small sample chamber 40, the plasma generator circuit 10 has a simplified configuration, as described in detail later, and occupies a small area, and this circuit configuration also contributes to providing the compact gas analyzer apparatus 1.

The internal pressure of the sample chamber (vessel) 40 is controlled to an appropriate negative pressure using the exhaust system 60 that is shared with the filter 20. An independent exhaust system, or an exhaust system shared with the process apparatus may be used instead. The internal pressure of the sample chamber 40 may be a pressure that facilitates the generation of the microplasma 8, and as one example, is in the range of 0.01 to 1 kPa. When the internal pressure of the process chamber 71 is managed or maintained so as to be around 1 to several hundred Pa, it is sufficient to manage the internal pressure of the sample chamber 40 to a lower pressure, for example, around 0.1 to several tens of Pa. The internal pressure may be managed to be 0.1 Pa or higher, 0.5 Pa or higher, 10 Pa or lower, or 5 Pa or lower. As one example, the inside of the sample chamber 40 may be depressurized to about 1-10 mTorr (or 0.13 to 1.3 Pa). By keeping the sample chamber 40 at the degree of depressurization given above, it becomes possible to generate the microplasma 8 at a low temperature using only the sample gas 9.

In the process monitoring system 70 with the gas analyzer apparatus 1, the monitoring target is the sample gas 9 supplied via the sampling apparatus 79 from the process chamber 71 where one or more plasma processes are carried out. Inside the sample chamber 40, by supplying RF power under appropriate conditions, it is possible to maintain the plasma 8 by merely introducing the sample gas 9 without using arc discharge or a plasma torch. By eliminating the need for a support gas such as argon gas, it is possible to generate the ionized plasma 8 with only (merely, simply) the sample gas 9 and analyze such ionized plasma 8 with analyzer 30. This means that it is possible to provide the gas analyzer apparatus 1, which has high measurement accuracy for the sample gas 9 and is also capable of quantitative measurement of components that are not limited to gas components. As a result, in the process monitor (process monitoring apparatus) 70 equipped with the gas analyzer apparatus 1, it is possible to stably and accurately monitor the internal state of the process chamber 71 of the process apparatus over a long period of time.

In process monitor 70, the plasma 8 of sample gas 9 is generated by sample chamber 40, which is independent of process chamber 71 and is dedicated to the analysis of gases. Accordingly, the microplasma 8 can be generated in the sample chamber 40 under conditions that are suited to sampling and gas analysis and differ to the conditions in the process chamber 71. For example, the internal state of the process chamber 71 can be monitored by converting the sample gas 9 into plasma (by plasmaized sample gas) even when no process plasma or cleaning plasma is being generated in the process chamber 71. The sample chamber 40 may be a small chamber (miniature chamber) with a size of several millimeters to several tens of millimeters, for example, suited to generating the microplasma 8. Due to the small capacity of the sample chamber 40, the entire analyzer apparatus 1 can be made compact and lightweight. It is possible to provide a gas analyzer apparatus 1 suited to real-time measurement. The gas analyzer apparatus 1 may be a portable or a handy type of device.

The gas analyzer apparatus 1 includes the control unit (central controller) 50 and the local controller 51. The central controller 50 may have the function of the local controller 51. The controller 50 includes a filter control unit (filter control function, filter controller or filter control apparatus) 53 that controls the filter unit (filter) 20, a detector control unit (detector control function, detector controller or detector control apparatus) 54 that controls the detectors 31 and 35, and a management control apparatus (management apparatus, management controller, manager, management function, or management unit) 55 for controlling data input/output, data analysis if required, and others of the gas analyzer apparatus 1. The controller 50 may have computer resources, including a memory 57 and a CPU 58, and the functions of the controller 50 may be provided by a program 59 recorded in the memory 57. The program (program product) 59 may be provided by recording the program on a suitable recording medium.

The local controller 51 includes a function 51a that controls on/off of the plasma generator circuit (plasma generation unit) 10, a function (plasma potential control unit, potential control apparatus, potential controller or voltage control apparatus) 51b that controls the voltage supplied to the control electrode 45 for controlling the floating potential of plasma 8, and a function 51c that controls the internal pressure of the sample chamber 40 using a pressure control valve 65 provided on a line connecting to the exhaust system 60. By controlling these factors, it is possible to stably generate the plasma 8 inside the sample chamber 40 by the self-tuning plasma generator circuit 10, even when the type of process carried out in the process chamber 71 has changed and/or the state of the process changes based on a request from the control unit 75 of the management system 70. Accordingly, the process monitoring apparatus 70, including the gas analyzer apparatus 1, can continuously analyze the sample gas 9 and monitor one or more processes.

The gas analyzer apparatus 1 includes a base 5 for integrating the sample chamber 40, the plasma generator 10, the analyzer 30, the controller 50 and 51 and the exhaust system 60 as one module. The base 5 may be a chassis or a base plate on which the sample chamber 40, the plasma generator 10, the analyzer 30, the controller 50 and 51, and the exhaust system 60 are assembled and may be installed or handled integrally as one module. The base 5 may be a housing (body) in which the sample chamber 40, the plasma generator 10, the analyzer 30, the controller 50 and 51, and the exhaust system 60 are housed and may be carried or installed on site integrally as a portable, movable, handy, laptop and/or desktop instrument. One of the typical sizes of the gas analyzer apparatus is 300-400 mm in height, 250-350 mm in wide, and 350-450 mm in depth, but not limited to.

The plasma inducing circuit 13 of the plasma generator circuit 10 generates the plasma 8 inside the sample chamber (plasma generator chamber) 40 using an electric field and/or a magnetic field applied through the dielectric wall structure 41 without using an electrode or a plasma torch. One example of the plasma inducing circuit 13 may include components or circuit elements, such as a coil and/or a capacitor for using a mechanism that excites the plasma 8 with high frequency (or radio frequency (RF)) power. The plasma inducing mechanism (method) may be Inductively coupled plasma (ICP), dielectric barrier discharge (DBD), electron cyclotron resonance (ECR), and others that use RF power.

FIG. 3 depicts an example of the system (gas analyzer apparatus) 1 that includes a plasma generator chamber (sample chamber) 40 and a plasma generator circuit 10. An example of the plasma generator circuit (plasma generating circuit, plasma generating unit, plasma generator) 10 includes the plasma inducing circuit 13 that includes at least one of a plasma inducing coil 13a and a capacitor 13b for inducing or generating a plasma in the sample chamber 40. The plasma generator circuit 10 also includes the RF power supply circuit 11 that is configured to supply RF power to the plasma inducing circuit 13. The plasma inducing circuit 13 is integrated directly (without using a transmission line) as at least a part of resonant components L2 and C2 of the RF power supply circuit 11.

The RF power supply circuit 11, that is the plasma generator circuit 10, may include a first inductor (L1) 12a coupled in series to a power source (first power supply); an electrically controlled switch 14, such as a MOSFET (power transistor, transistor switch), connected between the first inductor (L1) 12a and a primary common node 12b, the primary common node 12b connected to one of a second power supply or ground; a first capacitor (C1) 18 connected in parallel with the electrically controlled switch 14 and connected between the first inductor (L1) 12a and the primary common node 12b; a resonant circuit 16 connected in parallel with the electrically controlled switch 14. The resonant circuit 16 includes the plasma inducing circuit 13 and a third capacitor (C3) 17 coupled in series. The plasma inducing circuit 13 is integrated as at least a part of an inductor (second inductor, L2) 13a and/or a second capacitor (C2) 13b for directly configuring the resonant circuit 16. The RF power supply circuit 11 may further include a feedback circuit 15 for obtaining a feedback signal of the electrically controlled switch 14 from a feedback node 15a between the plasma inducing circuit 13 and the third capacitor C3. The feedback circuit 15 may include a base resistance (Rb) 15b. The RF power supply circuit 11 may include a bias circuit to apply Vbias 19.

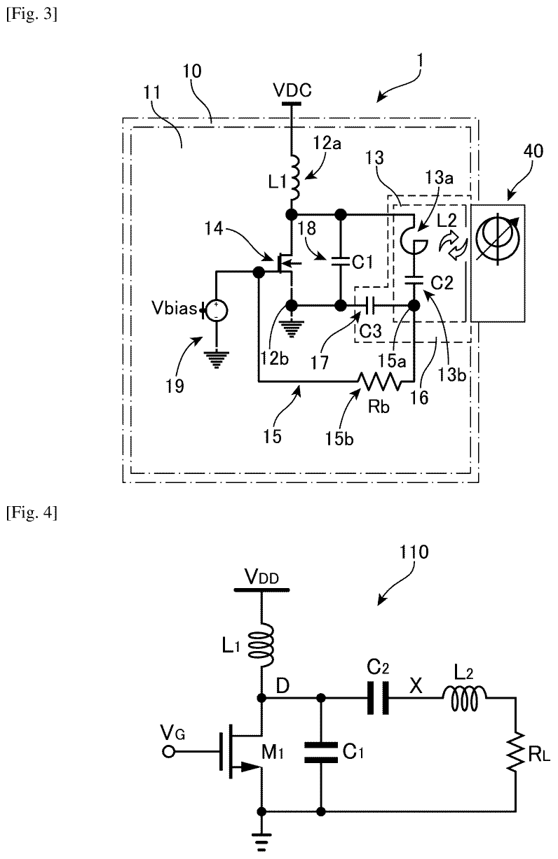

FIG. 4 depicts a typical self-tuned class-E power oscillator as a reference circuit of the above RF power supply circuit 11. Some types of class-E oscillators are described in US2020/0059198. The reference circuit 110 requires the Load (R_{L}) which will also need impedance matching with the rest of the circuit. The plasma generator circuit 10 with the RF power supply circuit 11 is different in a way that the inductor (L2) 13a and the capacitor (C2) 13b of the plasma inducing circuit 13 and the inductor (L2) 13a and the capacitor (C2) 13b of the LC resonant circuit 16 are common or identical respectively, and the inductor (L2) 13a and the capacitor (C2) 13b are integrated in the resonant circuit 16 as a part of the resonant circuit 16. That is the elements 13a and 13b of the plasma inducing circuit 13 directly configure the resonant circuit 16 (without transmission line), that is the load R_{L} and the connecting transmission line are eliminated, and the electrical parameters of the plasma generator chamber 40 becomes the property of the RF power supply circuit 11 (plasma generator circuit 10).

Hence, it can be possible to eliminate impedance matching, transmission lines, and a need for frequency reference completely. The plasma generator circuit 10 will operate continuously self-tuning to the load parameters as it changes, ensuring a continuous and reliable operation of the circuit as it adapts to everchanging load conditions automatically. In addition, because the plasma inducing circuit 13 is integrated into the RF power supply circuit 11 without a transmission line and any impedance matching mechanism is not required, the configuration of the plasma generator circuit 10 is simplified and the space required to install the plasma generator circuit 10 can be minimized. It contributes to the miniaturization of the gas analyzer apparatus 1, providing a modular, compact, and/or portable gas analyzer apparatus 1.

The plasma inducing circuit 13 may consist of the spiral planar inductor (L2) 13a and the capacitor (C2) 13b connected in series. The capacitor (C3) 17 (much larger in value than the capacitor C2) and the resistor Rb form the feedback circuit 15. Their values are calculated to ensure the appropriate phase shift for self-starting oscillation mode for generating plasma 8 in the chamber 40.

The plasma inducing circuit 13 is coupled (inductively and capacitively) to the plasma chamber 40, and the plasma chamber parameters change continuously during the regular (normal) operation, causing the effective parameters of the plasma inducing circuit 13 to change as well. The feedback circuit 15 ensures a continuous oscillation mode, tracking the plasma inducing circuit 13 effective (coupled) parameters, significantly reducing the overall system sensitivity to the component, environment, and plasma chamber variations. the feedback may be obtained from a node 15a between the plasma inducing circuit 13 and the capacitor (C3) 17, where signal amplitude is sufficiently high to feed for driving the electrically controlled switch 14. By integrating the plasma inducing circuit 13 as a part of the actual L-C parameters for designing the frequency of the oscillation of the RF power supply circuit 11, the frequency of the oscillation is self-determined by the effective L-C parameters. This self-adjustability of this design allows for not using a special impedance-matching circuitry and for robustness against any variations of chamber mechanical properties.

If the plasma inducing circuit is separated from the resonant circuit as a load R_{L} as shown in the reference circuit (Power Oscillator, PO) 110 depicted in FIG. 4, a cable (transmission line) is needed, and it has usually a 50-ohm impedance to connect the output of the PO 110 from R_{L} to the plasma inducing circuit installed at the plasma chamber 40. Because a cable (practically available) has a parameter called “characteristic impedance”, we have to make sure that the characteristic impedance of the plasma inducing circuit and PO 110 output (i.e., R_{L}) have exactly the same value, i.e., matched.

One of major differences from the reference circuit 110 of FIG. 4 is that a load R_{L} is not used because the plasma generator circuit 10 of our invention is for the Plasma Generator (integrated Plasma Generator). The reference circuit 110 uses a resonant circuit and R_{L} where R_{L} represents the Load of the Power Oscillator, the “Load” is whatever accepts the Rf power generated by the oscillator. In our invention for Integrated Plasma Generator 10, the resonant circuit 16 is used as a load, i.e. it also fulfills the function of R_{L}. Therefore, in our invention, the resonant circuit 16 becomes the load and is also a part of the plasma chamber 40. When plasma chamber parameters change (and they do in practical applications), so will the parameters of the resonant circuit 16 be leading to the integrated plasma generator self-tuning and maintaining the optimal performance automatically.

The term “Impedance matching” comes from transmission line engineering, i.e., it recognizes a “Source” generating Rf power, a “Load” accepting and dissipating this power, and a “Transmission line” as a means to connect the two and to deliver the power from “Source” to the “Load”. Now it is very important to match the physical parameters of all these elements (such as RF impedance) for the system to function properly.

In the case of Plasma Generation, there is a problem with the Plasma Chamber impedance changing all the time due to the changing conditions and causing the changes in the “Load” Rf impedance creating a mismatch. This causes many undesirable effects and can even cause circuit failure. As explained above, the plasma generator circuit 10 of this invention uses the Resonant circuit 16 as a “Load” i.e. the “Load” becomes integrated with the “Source” so no “Transmission line” is necessary to connect them. For the same reason of our Resonant circuit 16 being the “Load” and the “Source” at the same time, there is no need for the transmission line impedance matching, and because there is no separate “Source” and “Load” anymore, there is no need for a separate impedance matching between the two. In this invention, there is only one circuit (integrated into the RF power supply circuit (the Power Oscillator)), and when this circuit was properly designed and tested it showed a robust and efficient performance over the wide range of parameters of the Plasma Chamber 40.

As explained above, in this invention, a compact gas analyzer apparatus 1 with the plasma generator circuit 10 is provided. Because the plasma generator circuit 10 directly includes the plasma inducing circuit 13, when plasma chamber parameters change, the parameters of the resonant circuit 16 integrated in the plasma generator circuit 10 leads the plasma generator (plasma generator circuit 10 with the plasma inducing circuit 13) self-tuning and maintaining the optimal performance automatically. One of the best applications for this gas analyzer apparatus 1 is process monitoring/controlling as described above. The sample gas 9 to be measured is supplied via the sampling device (sampler) 79 from the process chamber 71 and the gas analyzer apparatus 1 analyzes the sample gas 9 via the generated plasma 8. The process controller 75 monitors and controls the processes 72 according to the measurement results of the gas analyzer apparatus 1. The applications of this gas analyzer apparatus 1 are not limited to the above, but are diverse, including environmental measurements at various sites and analysis of multiple (various) gases in multiple (various) situations, taking advantage of its compactness and portability.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

1-14. (canceled)

15. A gas analyzer apparatus comprising:
a sample chamber into which a sample gas to be measured flows;
a plasma generator circuit configured to generate a plasma in the sample chamber; and
an analyzer that is configured to analyze the sample gas via the plasma generated in the sample chamber,
wherein the plasma generator circuit includes a plasma inducing circuit that includes at least one of a plasma-inducing coil and a capacitor; and
an RF power supply circuit that is configured to supply RF power to the plasma inducing circuit, the plasma inducing circuit being integrated directly as at least a part of resonant components of the RF power supply circuit, and
wherein the RF power supply circuit includes:
an electrically controlled switch;
a first capacitor connected in parallel with the electrically controlled switch;
a resonant circuit connected in parallel with the electrically controlled switch that includes the plasma inducing circuit as an inductor and a second capacitor, and a third capacitor, the inductor, the second capacitor and the third capacitor being connected directly without a Load (R_{L}); and
a feedback circuit that is configured to obtain a feedback signal of the electrically controlled switch from a feedback node between the plasma inducing circuit and the third capacitor.

16. The gas analyzer apparatus according to claim 15, further comprising a base on which the sample chamber, the plasma generator circuit and the analyzer are assembled as one module.

17. The gas analyzer apparatus according to claim 16, further comprising an exhaust system that is configured to exhaust from the sample chamber and assembled on the base on which the sample chamber, the plasma generator circuit and the analyzer are assembled as one module.

18. The gas analyzer apparatus according to claim 15, further comprising an exhaust system that is configured to exhaust from the sample chamber; and
a base for integrating the sample chamber, the plasma generator circuit, the analyzer and the exhaust system as one module.

19. The gas analyzer apparatus according to claim 15, further comprising a first detector that is configured to detect filtered ionized gas from the plasma outputted from the sample chamber.

20. The gas analyzer apparatus according to claim 15, further comprising a second detector configured to analyze emission of ions in the plasma inside the sample chamber.

21. A system comprising:
the gas analyzer apparatus according to claim 15; and
a sampling device that is configured to supply the sample gas to be measured to the sample chamber.

22. The system according to claim 21, further comprising a process chamber in which a plasma process is performed and from which the sample gas is supplied to the gas analyzer apparatus.

23. The system according to claim 22, further comprising a process monitoring apparatus that monitors at least one process performed in the process chamber based on a measurement result of the gas analyzer apparatus.

24. The system according to claim 22, further comprising a process control apparatus that controls at least one process performed in the process chamber based on a measurement result of the gas analyzer apparatus.

25. A system comprising:
a plasma generator chamber; and
a plasma generator circuit, wherein the plasma generator circuit includes:
an electrically controlled switch;
a first capacitor connected in parallel with the electrically controlled switch;
a resonant circuit connected in parallel with the electrically controlled switch that includes a plasma inducing circuit for generating a plasma in the plasma generator chamber and a third capacitor, the plasma inducing circuit being integrated as an inductor and a second capacitor for directly configuring the resonant circuit, and the inductor, the second capacitor and the third capacitor being connected directly without a Load (R_{L}); and
a feedback circuit for obtaining a feedback signal of the electrically controlled switch from a feedback node between the plasma inducing circuit and the third capacitor.

26. The system according to claim 25 further comprising:
a sampler that is configured to supply a sample gas to be measured to the plasma generator chamber; and
an analyzer that analyzes the sample gas via a plasma generated in the plasma generator chamber.

27. The system according to claim 26,
wherein the sampler is configured to supply the sample gas delivered from a process to be monitored, and
the system further comprising a process monitor for monitoring and controlling the process according to measurement results of the analyzer.
